(19) United States
(12) Patent Application Publication
(10) Pub. No.: US 2026/0279561 A1
(43) Pub. Date: Sep. 17, 2026

(54) TECHNOLOGIES FOR PERFORMING DYNAMIC ALERT MANAGEMENT TO REDUCE CAREGIVER FATIGUE

(71) Applicant: Hill-Rom Services, Inc., Batesville, IN (US)

(72) Inventors: Sinan Batman, Batesville, IN (US); Georg Köllner, Batesville, IN (US); Michael S. Hood, Batesville, IN (US); Lori Ann Zapfe, Milroy, IN (US); Kelli F. Rempel, Chapel Hill, NC (US)

(73) Assignee: Hill-Rom Services, Inc., Batesville, IN (US)

(21) Appl. No.: 19/646,769

(22) Filed: Apr. 14, 2026

Related U.S. Application Data
(63) Continuation of application No. 17/671,671, filed on Feb. 15, 2022.
(60) Provisional application No. 63/159,106, filed on Mar. 10, 2021.

(51) Int. Cl. G16H 40/63

(52) U.S. Cl. CPC G16H 40/63

(57) ABSTRACT

Technologies for performing dynamic alert management to reduce caregiver fatigue may include a compute device. The compute device may include circuitry configured to receive alert data from a patient monitor device, to be provided to one or more caregivers in an alert. The circuitry may additionally be configured to determine whether alert suppression criteria indicative of one or more factors associated with alert-related fatigue is satisfied based on the received alert data. Additionally, the circuitry may be configured to suppress, in response to a determination that the alert suppression criteria is satisfied, the alert.

### CROSS-REFERENCE TO RELATED APPLICATIONS

This application is a continuation of U.S. application Ser. No. 17/671,671, filed on Feb. 15, 2022, now abandoned, which claimed the benefit, under 35 U.S.C. § 119(e), of U.S. Provisional Patent Application No. 63/159,106, filed on Mar. 10, 2021, the entirety of each of which is hereby expressly incorporated by reference herein.

### BACKGROUND

The present disclosure relates to managing alerts to caregivers pertaining to the condition of a patient, and more particularly to selectively suppressing alerts to reduce caregiver fatigue.

In a typical hospital setting, each patient is associated with one or more devices that is configured to produce an alert (e.g., an alarm) to be directed to caregivers, indicating a condition or status of the corresponding patient. For example, a patient bed may be capable of producing alerts associated with the position of one or more guard rails, the presence of moisture on a mattress under the patient, whether the patient is determined to be in the bed or out of the bed, and/or other factors associated with the present state of the patient. Additionally, the same patient may be monitored by various monitoring devices, such as a heart rate monitor and/or a breathing monitor. Further, one or more devices may be present in the room with the patient, capable of sending an alert at the direction of the patient, such as a nurse call device. As such, a soundscape analysis of a typical hospital setting would indicate that caregivers are inundated with alerts throughout a shift. Due to the abundance of alerts, caregivers may have difficulty interpreting the cause of an alert, next steps to be taken in response to an alert, and who is responsible for taking those next steps. Relatedly, due to fatigue from reviewing a continual stream of alerts, caregivers may inadvertently dismiss an alert that warrants additional attention.

### SUMMARY

The present application discloses one or more of the features recited in the appended claims and/or the following features which, alone or in any combination, may comprise patentable subject matter:

According to an aspect of the present disclosure, a compute device may include circuitry configured to receive alert data from a patient monitor device to be provided to one or more caregivers in an alert. The circuitry may be further configured to determine whether alert suppression criteria indicative of one or more factors associated with alert-related fatigue is satisfied based on the received alert data. Further, the circuitry may be configured to suppress, in response to a determination that the alert suppression criteria is satisfied, the alert.

In some embodiments, the circuitry may be configured such that determining whether the alert suppression criteria is satisfied includes determining whether the alert suppression criteria is satisfied based on a model of caregiver fatigue. In some embodiments, determining whether the alert suppression criteria is satisfied based on a model of caregiver fatigue may include determining whether the alert suppression criteria is satisfied based on a work schedule of each caregiver. The circuitry, in some embodiments, may be configured to determine whether to suppress the alert as a function of medical record data associated with a patient to whom the alert pertains. In some embodiments, determining whether alert suppression criteria is satisfied may include determining whether a defined alert suppression time period associated with the patient monitor device is satisfied. The circuitry, in some embodiments, may be configured to determine whether the alert suppression criteria is satisfied by determining whether a priority associated with the alert data satisfies a reference priority in the alert suppression criteria.

The circuitry of the compute device, in some embodiments, may be configured such that determining whether the alert suppression criteria is satisfied includes determining whether the alert suppression criteria is satisfied using a neural network having weights based on the alert suppression criteria. Additionally or alternatively, the circuitry may be configured to provide, in response to a determination that the alert suppression criteria is not satisfied, the alert indicative of the alert data to one or more of the caregivers. The circuitry may be further configured to select a subset of the caregivers to receive the alert based on a model of caregiver fatigue for each caregiver. Additionally or alternatively, the circuitry may be configured to select a subset of the caregivers to receive the alert as a function of a proximity of each caregiver to a patient associated with the monitor device that produced the alert data. In some embodiments, providing the alert may include providing the alert to a mobile communication device carried by one of the caregivers.

Additionally or alternatively, providing the alert may include providing the alert to a caregiver notification device installed in a hospital. In some embodiments, the circuitry may be configured such that providing the alert includes providing data indicative of a reason why the alert was provided. The circuitry of the compute device, in some embodiments, may be configured to provide, with the alert, data indicative of one or more steps to be performed to address the alert. Additionally or alternatively, the circuitry may be configured to determine a result of providing the alert to the caregiver and update the alert suppression criteria as a function of the determined result. In some embodiments, determining a result includes determining whether the alert was accepted or dismissed by a caregiver who received the alert. The circuitry, in some embodiments, may determine whether the alert was forwarded by a recipient caregiver to another caregiver. In some embodiments, the circuitry may be configured such that updating the alert suppression criteria includes providing the determined result to a neural network as training data.

The circuitry of the compute device, in some embodiments, may be configured such that updating the alert suppression data includes decreasing an assigned suppression value associated with the alert data if the alert was accepted. Updating the alert suppression data may include increasing an assigned suppression value associated with the alert data if the alert was dismissed. The circuitry may, in some embodiments, be configured to produce a report indicative of a set of patient monitor devices determined to have more dismissed alerts than other patient monitor devices.

In another aspect of the present disclosure, a method may include receiving, by a compute device, alert data from a patient monitor device to be provided to one or more caregivers in an alert. The method may additionally include determining, by the compute device, whether alert suppression criteria indicative of one or more factors associated with alert-related fatigue is satisfied based on the received alert data. Further, the method may include suppressing, by the compute device and in response to a determination that the alert suppression criteria is satisfied, the alert.

In some embodiments, determining whether the alert suppression criteria is satisfied may include determining whether the alert suppression criteria is satisfied based on a model of caregiver fatigue. Determining whether the alert suppression criteria is satisfied based on a model of caregiver fatigue may additionally or alternatively include determining whether the alert suppression criteria is satisfied based on a work schedule of each caregiver. The method may include determining, by the compute device, whether to suppress the alert as a function of medical record data associated with a patient to whom the alert pertains. In some embodiments, determining whether alert suppression criteria is satisfied may include determining whether a defined alert suppression time period associated with the patient monitor device is satisfied. In determining whether the alert suppression criteria is satisfied, the method, in some embodiments, may include determining whether a priority associated with the alert data satisfies a reference priority in the alert suppression criteria.

In some embodiments, determining whether the alert suppression criteria is satisfied may include determining whether the alert suppression criteria is satisfied using a neural network having weights based on the alert suppression criteria. The method, in some embodiments, may include providing, by the compute device and in response to a determination that the alert suppression criteria is not satisfied, the alert indicative of the alert data to one or more of the caregivers. Additionally or alternatively, the method may include selecting, by the compute device, a subset of the caregivers to receive the alert based on a model of caregiver fatigue for each caregiver. The method may include selecting, by the compute device, a subset of the caregivers to receive the alert as a function of a proximity of each caregiver to a patient associated with the monitor device that produced the alert data. Providing the alert, in some embodiments, may include providing the alert to a mobile communication device carried by one of the caregivers.

In some embodiments, providing the alert may include providing the alert to a caregiver notification device installed in a hospital. Additionally or alternatively, providing the alert may include providing data indicative of a reason why the alert was provided. Providing the alert, in some embodiments, may include providing data indicative of one or more steps to be performed to address the alert. In some embodiments, the method may include determining, by the compute device, a result of providing the alert to the caregiver and updating, by the compute device, the alert suppression criteria as a function of the determined result. Determining a result may, in some embodiments, include determining whether the alert was accepted or dismissed by a caregiver who received the alert. Additionally or alternatively, determining a result may include determining whether the alert was forwarded by a recipient caregiver to another caregiver.

In some embodiments, updating the alert suppression criteria includes providing the determined result to a neural network as training data. Updating the alert suppression data may include decreasing an assigned suppression value associated with the alert data if the alert was accepted. Conversely, updating the alert suppression data may include increasing an assigned suppression value associated with the alert data if the alert was dismissed. In some embodiments, the method may include producing a report indicative of a set of patient monitor devices determined to have more dismissed alerts than other patient monitor devices.

According to another aspect of the present disclosure, one or more machine-readable storage media may include instructions stored thereon that, in response to being executed, may cause a compute device to receive alert data from a patient monitor device to be provided to one or more caregivers in an alert. The instructions may further cause the compute device to determine whether alert suppression criteria indicative of one or more factors associated with alert-related fatigue is satisfied based on the received alert data. Further, the instructions may cause the compute device to suppress, in response to a determination that the alert suppression criteria is satisfied, the alert.

In some embodiments, determining whether the alert suppression criteria is satisfied may include determining whether the alert suppression criteria is satisfied based on a model of caregiver fatigue. Additionally or alternatively, determining whether the alert suppression criteria is satisfied based on a model of caregiver fatigue may include determining whether the alert suppression criteria is satisfied based on a work schedule of each caregiver. In some embodiments, the one or more instructions may cause the compute device to determine whether to suppress the alert as a function of medical record data associated with a patient to whom the alert pertains. Determining whether alert suppression criteria is satisfied may include determining whether a defined alert suppression time period associated with the patient monitor device is satisfied. In some embodiments, determining whether the alert suppression criteria is satisfied includes determining whether a priority associated with the alert data satisfies a reference priority in the alert suppression criteria.

Determining whether the alert suppression criteria is satisfied may include determining whether the alert suppression criteria is satisfied using a neural network having weights based on the alert suppression criteria. The one or more instructions may, in some embodiments, cause the compute device to provide, in response to a determination that the alert suppression criteria is not satisfied, the alert indicative of the alert data to one or more of the caregivers. Additionally or alternatively, the one or more instructions may cause the compute device to select a subset of the caregivers to receive the alert based on a model of caregiver fatigue for each caregiver. In some embodiments, the one or more instructions may cause the compute device to select a subset of the caregivers to receive the alert as a function of a proximity of each caregiver to a patient associated with the monitor device that produced the alert data.

Providing the alert, in some embodiments, may include providing the alert to a mobile communication device carried by one of the caregivers. In some embodiments, providing the alert includes providing the alert to a caregiver notification device installed in a hospital. Additionally or alternatively, providing the alert includes providing data indicative of a reason why the alert was provided. Providing the alert, in some embodiments, may include providing data indicative of one or more steps to be performed to address the alert. The one or more machine-readable storage media may include one or more instructions that cause the compute device to determine a result of providing the alert to the caregiver and update the alert suppression criteria as a function of the determined result. In some embodiments, determining a result includes determining whether the alert was accepted or dismissed by a caregiver who received the alert. Determining a result may include determining whether the alert was forwarded by a recipient caregiver to another caregiver. In some embodiments, updating the alert suppression criteria includes providing the determined result to a neural network as training data. In some embodiments, updating the alert suppression data may include decreasing an assigned suppression value associated with the alert data if the alert was accepted. Conversely, updating the alert suppression data may include increasing an assigned suppression value associated with the alert data if the alert was dismissed. In some embodiments, the one or more instructions may cause the compute device to produce a report indicative of a set of patient monitor devices determined to have more dismissed alerts than other patient monitor devices.

Additional features, which alone or in combination with any other feature(s), such as those listed above and/or those listed in the claims, may comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a diagram of a system for dynamically managing patient alerts to reduce alert-related fatigue of caregivers;

FIG. 2 is a diagram of components of a compute device included in the system of FIG. 1;

FIGS. 3-7 are diagrams of at least one embodiment of a method for dynamically managing patient alerts that may be performed by the system of FIG. 1; and

FIG. 8 is a diagram of a workflow that may be implemented by the system of FIG. 1 for dynamically managing patient alerts.

### DETAILED DESCRIPTION

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one of A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

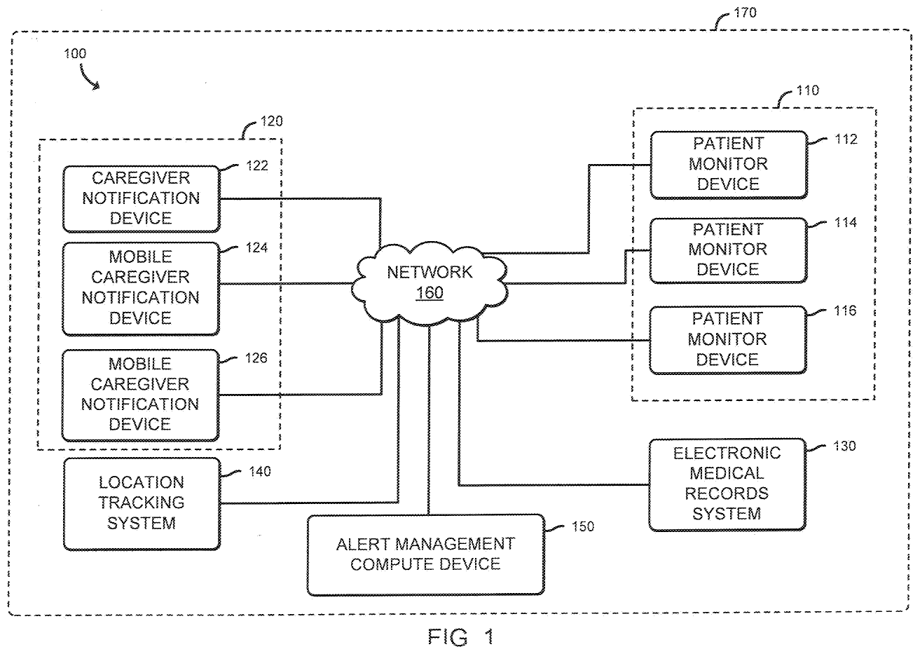

Referring now to FIG. 1, a system 100 for dynamically managing patient alerts to reduce alert-related caregiver fatigue includes a set of patient monitor devices 110, 112, 114, 116, a set of caregiver notification devices 120, 122, 124, 126, an electronic medical records (EMR) system 130, a location tracking system 140 (e.g., a set of devices, such as near field communication (NFC) tags (e.g., worn by caregivers) and tag readers (e.g., located throughout the clinical environment 170) that communicate to track the locations of caregivers), and an alert management compute device 150. The system 100, in the illustrative embodiment, operates in a clinical environment 170 (e.g., a hospital, a nursing home, etc.) in which patients are cared for by caregiver (e.g., nurses, doctors, etc.). The patient monitor devices 110 may be embodied as any device capable of detecting a status associated with a patient and sending alert data indicative of an alert associated with the patient (e.g., if the determined status satisfies a reference status). As such, the patient monitor devices 110 may include pulse oximeters, heart rate monitors, breathing monitors, incontinence event detectors, patient position detectors, patient support apparatus position detectors, guard rail position detectors, patient fall detectors, anesthesia machines, feeding pumps, wound vacuums, compression pumps, etc. Additionally, the patient monitor devices 110 may include devices affirmatively activated by a corresponding patient to send an alert, such as a nurse call device that enables the patient to request assistance from a caregiver.

The caregiver notification devices 120, 122, 124, 126 may be embodied as any devices configured to provide notifications of the alerts (e.g., from the patient monitor devices 110) to caregivers. Some caregiver notification devices (e.g., the caregiver notification device) 122 are installed in the clinical environment 170 (e.g., in a patient room) while others, such as the mobile caregiver notification devices 124, 126, are mobile and carried by the caregivers. As such, the caregiver notification device 122 may be embodied as a digital whiteboard or other display device, a device capable of producing audible notifications (e.g., a speaker), a set of one or more color-coded lights, or similar device that is not typically moved from one location in the clinical setting to another location, while the mobile caregiver notification devices 124, 126 may be embodied as cellular phones, tablet compute devices, wearable compute devices (e.g., smart watches), notebook compute devices, personal digital assistants, or similar devices carried by caregivers.

The alert management compute device 150, in operation, operates as a hub between the patient monitor devices 110 and the caregiver notification device 120 and applies criteria to determine whether to suppress an alert or, if not suppress the alert completely, then selectively route the alert to particular caregiver notification devices, to reduce the amount of fatigue that caregivers would otherwise experience from receiving all alerts produced by all of the patient monitor devices throughout their shifts. In doing so, the alert management compute device 150, in the illustrative embodiment, may utilize electronic medical record data from an electronic medical records system 130 to determine whether certain information about a patient to whom an alert pertains increases or decreases the risk associated with an alert (e.g., a heightened heart rate associated with a patient known to have heart disease may present a higher risk than a patient without heart disease, a patient fall alert may be a higher risk for a patient that is older than a predefined age than for a younger patient, etc.). The alert management compute device 150, in the illustrative embodiment additionally or alternatively utilizes location data indicative of the locations of caregivers within the clinical environment 170 in determining how to route a given alert. For example, the alert management compute device 150 may determine the proximity of each caregiver to the patient to whom an alert pertains, determine whether a caregiver is preoccupied with another patient, and/or make other determinations based on the location data to define the route for the alert. Further, the alert management compute device 150, takes into account the amount of time that each caregiver has been working, information indicating their energy or activity levels, the amount of time that has elapsed since a particular patient monitor device 110 has sent an alert, caregiver responses (e.g., accepting, dismissing, forwarding, silencing, etc.) to alerts that have previously been sent to the caregiver notification devices 120, and/or other factors in determining how to manage incoming alerts from the patient monitor devices 110. In doing so, the alert management compute device 150 identifies updates that can be applied to the set of rules (e.g., criteria) defining how alerts of different types should be managed in different contexts, to further reduce alert-related fatigue among the caregivers. Accordingly, the system 100 enables caregivers to focus on alerts that they receive, with the understanding that those specific alerts are more likely to warrant their attention than alerts that they might receive in a system not equipped with the alert management compute device 150.

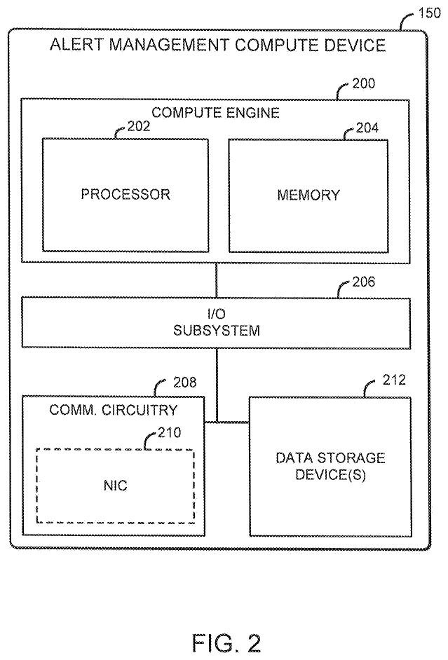

Referring now to FIG. 2, the illustrative alert management compute device 150 includes a compute engine 200, an input/output (I/O) subsystem 206, communication circuitry 208, and a data storage subsystem 212. Of course, in other embodiments, the alert management compute device 150 may include other or additional components, such as those commonly found in a computer (e.g., a display, peripheral devices, etc.). Additionally, in some embodiments, one or more of the illustrative components may be incorporated in, or otherwise form a portion of, another component.

The compute engine 200 may be embodied as any type of device or collection of devices capable of performing various compute functions described below. In some embodiments, the compute engine 200 may be embodied as a single device such as an integrated circuit, an embedded system, a field-programmable gate array (FPGA), a system-on-a-chip (SOC), or other integrated system or device. Additionally, in the illustrative embodiment, the compute engine 200 includes or is embodied as a processor 202 and a memory 204. The processor 202 may be embodied as any type of processor capable of performing the functions described herein. For example, the processor 202 may be embodied as a single or multi-core processor(s), a microcontroller, or other processor or processing/controlling circuit. In some embodiments, the processor 202 may be embodied as, include, or be coupled to an FPGA, an application specific integrated circuit (ASIC), reconfigurable hardware or hardware circuitry, or other specialized hardware to facilitate performance of the functions described herein.

The main memory 204 may be embodied as any type of volatile (e.g., dynamic random access memory (DRAM), etc.) or non-volatile memory or data storage capable of performing the functions described herein. Volatile memory may be a storage medium that requires power to maintain the state of data stored by the medium. In some embodiments, all or a portion of the main memory 204 may be integrated into the processor 202. In operation, the main memory 204 may store various software and data used during operation such as rules for suppressing and routing alerts, received alert data, caregiver location data, patient medical record data, caregiver activity level data, responses to previous alerts sent to caregivers, caregiver notification device identifiers, applications, libraries, and drivers.

The compute engine 200 is communicatively coupled to other components of the alert management compute device 150 via the I/O subsystem 206, which may be embodied as circuitry and/or components to facilitate input/output operations with the compute engine 200 (e.g., with the processor 202 and the main memory 204) and other components of the alert management compute device 150. For example, the I/O subsystem 206 may be embodied as, or otherwise include, memory controller hubs, input/output control hubs, integrated sensor hubs, firmware devices, communication links (e.g., point-to-point links, bus links, wires, cables, light guides, printed circuit board traces, etc.), and/or other components and subsystems to facilitate the input/output operations. In some embodiments, the I/O subsystem 206 may form a portion of a system-on-a-chip (SoC) and be incorporated, along with one or more of the processor 202, the main memory 204, and other components of the alert management compute device 150, into the compute engine 200.

The communication circuitry 208 may be embodied as any communication circuit, device, or collection thereof, capable of enabling communications over a network between the alert management compute device 150 and another device (e.g., the patient monitor devices 110, the caregiver notification devices 120, the electronic medical records system 130, the location tracking system 140, etc.). The communication circuitry 208 may be configured to use any one or more communication technology (e.g., wired or wireless communications) and associated protocols (e.g., Ethernet, Wi-Fi®, WiMAX, Bluetooth®, cellular, etc.) to effect such communication.

The illustrative communication circuitry 208 includes a network interface controller (NIC) 210. The NIC 210 may be embodied as one or more add-in-boards, daughter cards, network interface cards, controller chips, chipsets, or other devices that may be used by the alert management compute device 150 to connect with another compute device (e.g., the patient monitor devices 110, the caregiver notification devices 120, the electronic medical records system 130, the location tracking system 140, etc.). In some embodiments, the NIC 210 may be embodied as part of a system-on-a-chip (SoC) that includes one or more processors, or included on a multichip package that also contains one or more processors. In some embodiments, the NIC 210 may include a local processor (not shown) and/or a local memory (not shown) that are both local to the NIC 210. In such embodiments, the local processor of the NIC 210 may be capable of performing one or more of the functions of the compute engine 200 described herein. Additionally or alternatively, in such embodiments, the local memory of the NIC 210 may be integrated into one or more components of the alert management compute device 150 at the board level, socket level, chip level, and/or other levels.

Each data storage device 212, may be embodied as any type of device configured for short-term or long-term storage of data such as, for example, memory devices and circuits, memory cards, hard disk drives, solid-state drives, or other data storage device. Each data storage device 212 may include a system partition that stores data and firmware code for the data storage device 212 and one or more operating system partitions that store data files and executables for operating systems. While shown as a single unit, it should be appreciated that the components of the alert management compute device 150 may, in some embodiments, be distributed across multiple physical locations (e.g., multiple racks in a data center). Further, one or more of the components may be virtualized (e.g., in a virtual machine executing on one or more physical compute devices).

The patient monitor devices 110, the caregiver notification devices 120, the electronic medical records system 130, and the location tracking system 140 may have components similar to those described in FIG. 2 with reference to the alert management compute device 150. The description of those components of the alert management compute device 150 is equally applicable to the description of components of the patient monitor devices 110, the caregiver notification devices 120, the electronic medical records system 130, and the location tracking system 140. Further, it should be appreciated that any of the devices 110, 120, 150 and systems 130, 140 may include other components, sub-components, and devices commonly found in computing devices, which are not discussed above in reference to the alert management compute device 150 and not discussed herein for clarity of the description.

In the illustrative embodiment, the alert management compute device 150, the devices 110, 120, and the systems 130, 140 are in communication via a network 160, which may be embodied as any type of wired or wireless communication network, including local area networks (LANs) or wide area networks (WANs), digital subscriber line (DSL) networks, cable networks (e.g., coaxial networks, fiber networks, etc.), cellular networks (e.g., Global System for Mobile Communications (GSM), Long Term Evolution (LTE), Worldwide Interoperability for Microwave Access (WiMAX), 3G, 4G, 5G, etc.), radio area networks (RAN), global networks (e.g., the internet), or any combination thereof, including gateways between various networks.

In operation, the system 100 (e.g., the alert management compute device 150), in the illustrative embodiment, collects raw data from all medical devices (e.g., patient monitor devices 110) and an electronic medical records system (e.g., the electronic medical records system 130). Additionally, the system 100 (e.g., the alert management compute device 150) executes predictive algorithms to determine a subset of actionable alerts personalized to each patient. Further, the system 100 (e.g., the alert management compute device 150) executes algorithms to determine which caregiver(s) to inform about an alert, when, and how to inform the caregiver(s) of the alert. Alerts sent to patient notification devices 120 may be silent, checked on a mobile device, and/or interpreted through a digital whiteboard. Further, upon request, the system 100 informs a caregiver about the next steps to take in response to an alert. Key broader stakeholder benefits include one system to manage the flow of data, alerting, and the ability to gain insight about patient satisfaction, changes in retention, and other metrics over time via reporting. The alert management compute device 150, in the illustrative embodiment, integrates with EMR and patient, device, and caregiver locator services. Additionally, the alert management compute device 150 provides smart integration with medical devices, sensing disruptive third-party interface changes. The alert management compute device 150 illustratively operates as a central hub that integrates and communicates the following to existing mobile platforms and/or EMR: i) notification of actionable personalized patient risk and trends, ii) next caregiver actions and their priority across all patients under a caregiver's responsibility, and iii) clickable links to access rationale for why an alert was provided and for the next suggested action (i.e., interpretability).

Additionally, the alert management compute device 150, in the illustrative embodiment, utilizes a continual learning algorithm that observes context and provides caregiver-dependent muting and unmuting of alerts. Further, the alert management compute device 150 performs intelligent task delegation to specific person(s) by considering caregiver context (e.g., distance to patient, etc.). Additionally, the system enables easy close out of tasks to document in EMR and integrates with a digital whiteboard system and a mobile communication device platform (e.g., Hillrom Voalte® from Hillrom of Batesville, IN). The alert management compute device 150, in operation, provides improved patient outcomes over typical systems, and may improve associated hospital ranking and reimbursement rates by reducing caregiver reaction times and distractions away from patients at higher risk of fall and personal injury. The alert management compute device 150 may also reduce injuries and death associated with dismissing high priority alerts. Further, the alert management compute device 150 may reduce sleep interruptions of as a result of providing a less-noisy environment. Relatedly, the alert management compute device 150 may reduce patient stress from being subject to dissonant and loud alerts. The alert management compute device 150 may provide improved caregiver and patient satisfaction by providing a less noisy and peaceful environment. The system 100 enables caregiver to deliver better patient care by enabling caregiver to always be working on the most important tasks, having clear accountability to execute tasks, and having easily interpretable causes of alerts and information on the next steps to be taken (e.g., to respond to an alert). The system 100 provides improved caregiver productivity, satisfaction, longevity, and retention by reducing alert fatigue and distractions and, by offloading decision making (e.g., regarding whether alerts warrant further attention), improves each caregiver's focus and energy.

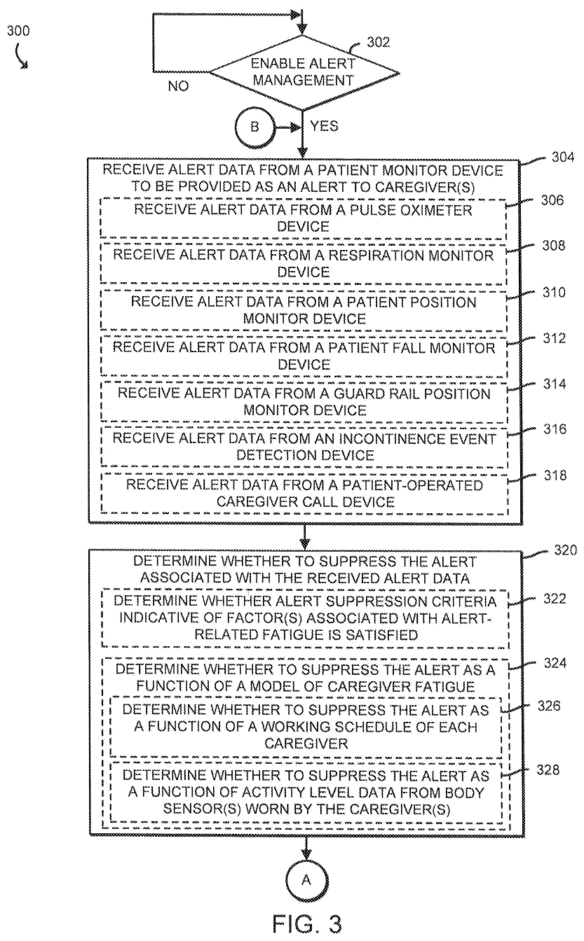

Referring now to FIG. 3, the system 100, and in particular, the alert management compute device 150, may perform a method 300 for dynamically managing patient alerts. In the illustrative embodiment, the method 300 begins with block 302, in which the alert management compute device 150 determines whether to enable alert management. In doing so, the alert management compute device 150 may determine to enable alert management in response to a determination that a configuration setting (e.g., in the memory 204) indicates to do so, in response to a determination that the alert management compute device 150 is communicatively connected to one or more of the patient monitor devices 110, and/or based on other factors. Regardless, in response to a determination to enable alert management, the method 300 advances to block 304, in which the alert management compute device 150 receives (e.g., through the network 160) alert data (e.g., any data or other signal indicative of an alert) from a patient monitor device 110 to be provided as an alert to one or more caregivers (e.g., via one or more of the caregiver notification devices 120). In doing so, and as indicated in block 306, the alert management compute device 150 may receive alert data (e.g., oxygen saturation of a patient's blood and heart rate) from a pulse oximeter device, as indicated in block 306. As indicated in block 308, the alert management compute device 150 may receive alert data (e.g., a breathing rate of a patient) from a respiration monitor associated with a patient in the clinical environment 170. In some embodiments, the alert management compute device 150 may receive alert data (e.g., a change in a support apparatus position, such as an inclination of a portion of a patient bed, an indication that the patient has shifted their weight from one portion of a patient bed to another portion of the patient bed, etc.) from a patient position monitor device, as indicated in block 310. In receiving alert data, the alert management compute device 150 may receive alert data from a patient fall monitor device (e.g., data indicating that the patient may have fallen off of a support apparatus, such as data indicative of a sudden change in the amount of weight supported by the apparatus), as indicated in block 312. Relatedly, the alert management compute device 150 may receive alert data from a guard rail position monitor device (e.g., data indicating that a guard rail on a patient bed has been lowered from a raised position), as indicated in block 314. In some embodiments, the alert management compute device 150 may receive alert data from an incontinence event detection device (e.g., data indicating that moisture has been sensed beneath a patient resting on a patient bed), as indicated in block 316. The alert management compute device 150 may additionally or alternatively receive alert data from a patient-operated caregiver call device (e.g., a request for assistance initiated by the patient by pressing a nurse call button, speaking into a pillow speaker, requesting assistance through a compute device located in the patient's room, etc.), as indicated in block 318.

After receiving alert data from a patient monitor device 110, the method 300 advances to block 320, in which the alert management compute device 150 determines whether to suppress the alert (e.g., prevent the alert from being sent to a caregiver notification device 120) associated with the received alert data. In doing so, and as indicated in block 322, the alert management compute device 150, in the illustrative embodiment, determines whether alert suppression criteria indicative of one or more factors associated with alert-related fatigue is satisfied. As described herein, the determination, in the illustrative embodiment, is made based on a combination of factors, rather than a single dispositive factor. As indicated in block 324, the alert management compute device 150 may determine whether to suppress the alert as a function of a model of caregiver fatigue. In doing so, and as indicated in block 326, the alert management compute device 150 may determine whether to suppress the alert as a function of a working schedule of each caregiver. That is, if an alert that is determined by the alert management compute device 150 to be of low risk (e.g., that the inclination of a patient's bed has changed by a threshold amount), the alert management compute device 150 may determine to suppress the alert if the caregivers in the clinical environment 170 are near the end of their shifts and more likely to have fatigue from responding to previous alerts than if the caregivers are near the beginning of their shifts. Relatedly, and as indicated in block 328, the alert management compute device 150 may determine whether to suppress the alert as a function of activity level data from one or more body sensors (e.g., a pedometer, a heart rate sensor, etc.) worn by the caregivers. For example, the alert management compute device 150 may determine periods of time when caregivers have higher energy levels (e.g., as indicated by the activity level data) and be more likely to not to suppress an alert during a period of relatively high energy or activity of the caregivers.

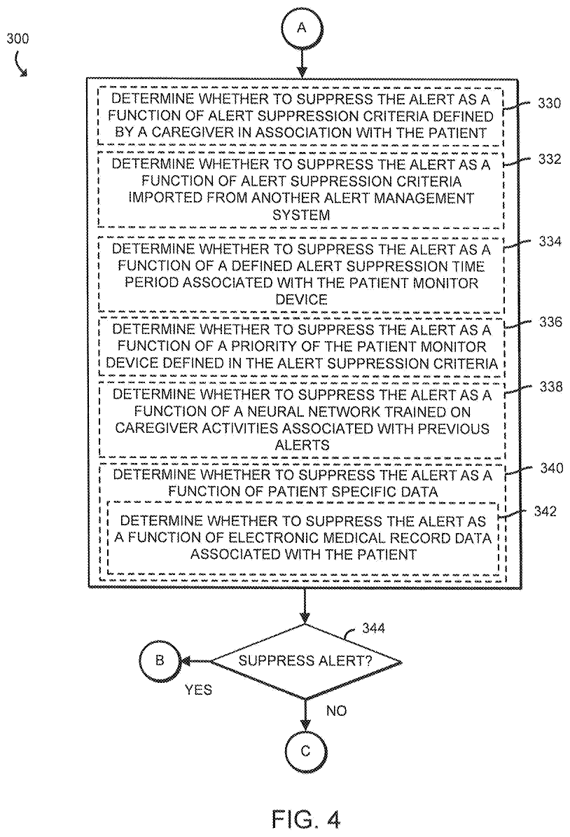

Referring now to FIG. 4, as indicated in block 330, the alert management compute device 150, in determining whether to suppress the alert, may determine whether to suppress the alert as a function of alert suppression criteria defined by a caregiver in association with the patient (e.g., the patient to whom the alert data pertains). That is, in some embodiments, the alert management compute device 150 may utilize a set of alert suppression criteria that is specific to a particular patient and was defined by a particular caregiver (e.g., by providing, through a user interface, a set of rules that define how an alert pertaining to that patient should be managed). As indicated in block 332, the alert management compute device 150 may determine whether to suppress the alert as a function of alert suppression criteria imported from another alert management system. That is, in some embodiments, a set of criteria that has been determined to be particularly effective in directing relevant alerts to caregivers while suppressing less relevant alerts may be exported from one alert management system and imported into the alert management system 100 (e.g., into the memory 204 or data storage 212 of the alert management compute device 150).

In some embodiments, the alert management compute device 150 may make the determination of whether to suppress the alert as a function of a defined alert suppression time period associated with the patient monitor device that sent the alert data to the alert management compute device 150, as indicated in block 334. That is, the alert management compute device 150 may prevent multiple alerts from the same patient monitor device 110 from being sent to caregiver notification device(s) 120 during a given time period (e.g., allow only one alert from the patient monitor device 112 for every five minute interval) to reduce the possibility of alerts from one patient monitor device outnumbering alerts from other patient monitor devices 110 that may be of higher importance (e.g., representing a more urgent status of a patient). In some embodiments, the alert management compute device 150 may determine whether to suppress an alert as a function of a priority of a patient monitor device 110, as indicated in block 336. The priority may be defined in the alert suppression criteria (e.g., in association with an identifier, such as an internet protocol (IP) address, media access control (MAC) address, or other unique identifier of the patient monitor device 110 that sent the alert data). That is, the alert management compute device 150 may determine not to suppress an alert if the associated patient monitor device 110 has a relatively high priority (e.g., satisfying a target priority defined in the alert suppression criteria) as compared to a similar situation in which the patient monitor device 110 has a lower priority.

As indicated in block 338, the alert management compute device 150 may determine whether to suppress the alert as a function of a neural network trained on caregiver activities associated with previous alerts. That is, the alert suppression criteria may be in whole or in part embodied as a neural network (e.g., as a data structure in the memory 204 of the alert management compute device) in which the value(s) of one or more weights that influence an output (e.g., an inference or determination) made by the neural network are determined based on feedback (e.g., responses) from caregivers to alerts that were not suppressed. For example, if an alert of a certain type (e.g., as defined by the alert data, such as the identity of patient monitor device 110 that sent the alert data, a value of a monitored condition (e.g., heart rate, bed angle, etc.), etc.) was sent to one or more caregiver notification devices 120 and was dismissed or silenced by the receiving caregiver(s), the response (e.g., dismissal of the alert) may be used to train the neural network to be more likely to suppress an alert of the same type in the future. Conversely, if the alert was accepted, the neural network may be trained to be biased towards not suppressing a later alert of the same type.

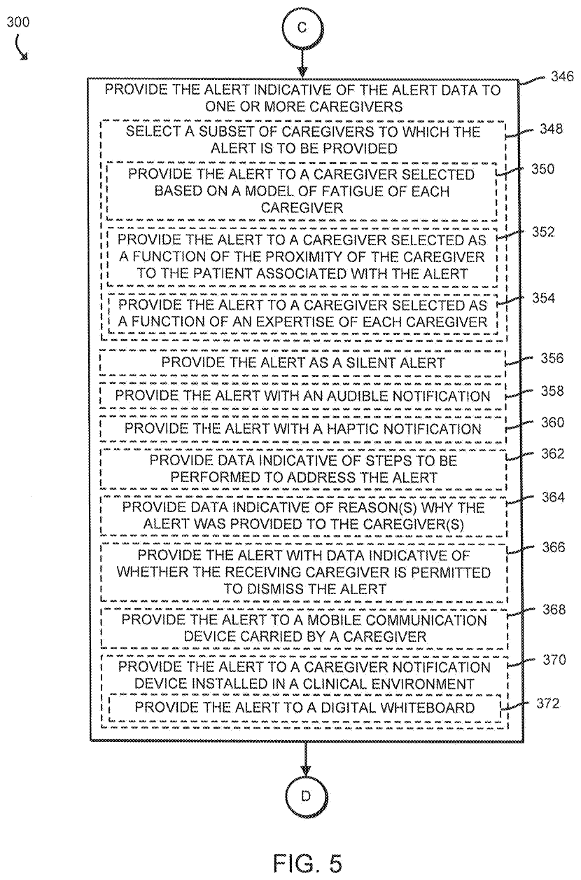

Still referring to FIG. 4, the alert management compute device 150 may determine whether to suppress the alert as function of patient specific data, as indicated in block 340. For example, and as indicated in block 342, the alert management compute device 150 may determine whether to suppress the alert as a function of electronic medical record data associated with the patient to whom the alert data pertains. That is, in some embodiments, the alert data may include a patient identifier or other information associated with patient identifier (e.g., patient room number) that is usable by the alert management compute device 150 to query the electronic medical record system 130 for risk factors (e.g., that the patient is above a threshold age, that the patient has heart disease, etc.) that would increase the risk associated with the alert data (e.g., that the patient may have fallen out of the bed, that the patient has a heightened heart rate, etc.). If a relevant risk factor is present, the alert management compute device 150 may bias the determination of whether to suppress the alert in favor or not suppressing the alert. In block 344, the alert management compute device 150 determines the subsequent course of action based on whether the alert is to be suppressed or not (i.e., based on the determination made in block 320). In response to a determination that the alert is to be suppressed, the method 300 loops back to block 304 of FIG. 3, in which the alert management compute device 150 may receive additional alert data (e.g., associated with another alert). Otherwise, if the alert management compute device 150 has determined not to suppress the alert, the method 300 advances to block 346 of FIG. 5, in which the alert management compute device 150 provides the alert indicative of (e.g., associated with) the alert data to one or more caregivers (e.g., to one or more of the caregiver notification devices 120).

Referring now to FIG. 5, in providing the alert to one or more caregivers, the alert management compute device 150, in the illustrative embodiment, selects a subset of all of the caregivers in the clinical environment 170 to which the alert is to be provided, as indicated in block 348. In doing so, the alert management compute device 150, in the illustrative embodiment, provides the alert based on a model of fatigue of each caregiver (e.g., the model from block 324 of FIG. 3), as indicated in block 350. That is, the alert management compute device 150 excludes, from the subset, any caregivers determined to be fatigued beyond a reference amount. As indicated in block 352, in selecting a subset of the caregivers, the alert management compute device 150 may determine to provide the alert to one or more caregivers as a function of (e.g., based at least in part on) each caregiver's proximity to the patient associated with the alert. For example, the alert management compute device 150 may select one or more caregivers determined to be within a predefined range of the patient's room (e.g., determined based on location data from the location tracking system 140). Additionally or alternatively, the alert management compute device 150 may determine to provide the alert to one or more caregivers selected as a function of an expertise of each caregiver (e.g., as defined in a dataset, in the memory 204 and/or in the data storage 212, of caregiver identifiers and associated areas of expertise), as indicated in block 354. For example, if the patient to whom the alert pertains is older than a reference age, the alert management compute device 150 may narrow the subset of caregivers to those having an educational background and/or threshold number of years of experience in geriatrics.

In providing the alert to one or more caregivers, the alert management compute device 150 may define how the alert is to be provided (e.g., with data indicative of how the alert is to be presented by the receiving caregiver notification device 120). In doing so, the alert management compute device 150 may provide the alert as a silent alert (e.g., only having a visual component, such as text and/or graphics), as indicated in block 356. Additionally or alternatively, the alert management compute device 150 may provide the alert with an audible notification (e.g., a sound indicative of the priority of the alert, a sound indicative of the type of patient monitor device 110 that originally produced the alert data, a spoken description of the alert, etc.), as indicated in block 358. In some embodiments, the alert management compute device 150 may provide the alert with a haptic notification (e.g., vibration), as indicated in block 360. In some embodiments, the alert management compute device 150 may supplement the alert data received from the patient monitor device 110 with data indicative of one or more steps to be performed (e.g., by a caregiver who accepts the alert and is presented with the step(s) to be performed) to address the alert, as indicated in the block 362. The data indicative of the step(s) to be performed to address the alert may be accessed, by the alert management compute device 150, from a data set (e.g., in the memory 204 or in the data storage 212). In other embodiments, the alert data received from the patient monitor device 110 already includes data indicative of the step(s) to be performed to address the alert.

In some embodiments, the alert management compute device 150 may provide (e.g., with the alert to a caregiver notification device 120 and/or to another compute device, such as a technical administrative compute device), data indicative of reason(s) why the alert was provided to the caregiver(s), as indicated in block 364. The data may identify one or more rules that were satisfied and that lead to the determination to send the alert, the selection of the caregiver(s) to whom the alert was sent, and reasons for presenting the alert in a particular way (e.g., with a haptic notification because the target caregiver is likely in a loud environment based on the determined location of the caregiver reported by the location tracking system 140). In some embodiments, the alert management compute device 150 may provide the alert with data indicative of whether a receiving caregiver is permitted to dismiss (e.g., reject) the alert, as indicated in block 366. For example, if the alert management compute device 150 determines that the alert satisfies a threshold priority (e.g., due to a risk associated with the alert) and the alert is being sent to only one caregiver, the alert management compute device 150 may include data indicating to the corresponding caregiver notification device(s) 110 that the caregiver is not permitted to dismiss the alert.

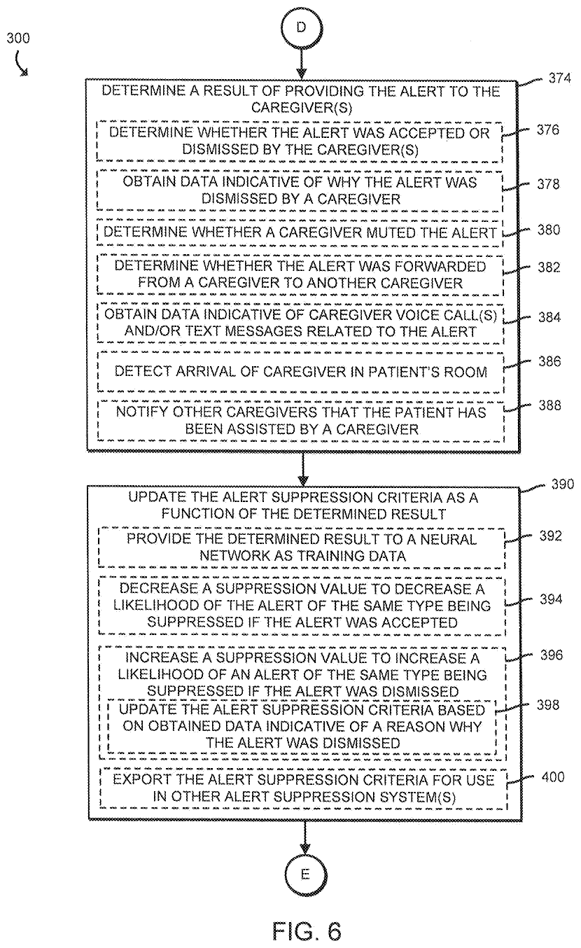

As indicated in block 368, in providing the alert, the alert management compute device 150 may provide the alert to a mobile communication device (e.g., phone, tablet, pager, etc.) carried by a caregiver, such as one or more of the mobile caregiver notification devices 124, 126. Additionally or alternatively, the alert management compute device 150 may provide the alert to a caregiver notification device 120 that is installed in the clinical environment 170 (e.g., a patient room, in a hallway, etc.), as indicated in block 370. For example, and as indicated in block 372, the alert management compute device 150 may provide the alert to a digital whiteboard (e.g., the caregiver notification device 122). Subsequently, the method 300 advances to block 374 of FIG. 6, in which the alert management compute device 150 determines a result of providing the alert to the caregiver(s) (e.g., to the caregiver(s) selected in block 348).

Referring now to FIG. 6, in determining a result of providing the alert, the alert management compute device 150 may determine whether the alert was accepted or dismissed by the caregiver(s) (e.g., the caregiver(s) to whom the alert was sent), as indicated in block 376. That is, the alert management compute device 150 may receive, from a caregiver notification device 120, response data which may be embodied as any indicative of a response provided by the caregiver after being presented with the alert. For example, the caregiver may dismiss (e.g., reject) the alert, mute (e.g., silence) the alert if it has an audible notification, forward the alert to another caregiver, or accept the alert. In some embodiments, the alert management compute device 150 may additionally receive data indicative of a reason why the alert was dismissed (e.g., a textual reason input by the caregiver on a mobile caregiver notification device 124, 126 explaining why the alert was not accepted), as indicated in block 378.

As indicated in block 380, the alert management compute device 150 may determine whether the caregiver muted the alert. Similarly, and as indicated in block 382, the alert management compute device 150 may determine whether the caregiver forwarded the alert to another caregiver. In some embodiments, the alert management compute device 150 may obtain (e.g., from a caregiver notification device, such as a mobile caregiver notification device 124, 126) data indicative of caregiver voice calls and/or text messages related to the alert, as indicated in block 384. That is, a caregiver notification device 124, 126 may present, to a caregiver, a notification of an alert, track whether the caregiver placed a call or sent a text message based on the notification of the alert (e.g., due to the caregiver selecting an option to place a call or send a text message in a user interface presented in connection with the alert), and send data indicative of the placed call or text message to the alert management compute device 150. In some embodiments, the alert management compute device 150 may detect the arrival of a caregiver (e.g., who received the alert) in the patient's room (e.g., based on location tracking data from the location tracking system 140). Relatedly, in some embodiments, the alert management compute device 150 may record the amount of time that elapses between the sending of the alert to a caregiver and the arrival of the caregiver in a patient's room (e.g., for use in analyzing of the responsiveness of the caregiver). As indicated in block 388, the alert management compute device 150 may send a notification to other caregivers (e.g., to corresponding caregiver notification devices 120) that the patient associated with the alert has been assisted by a caregiver (e.g., in response to detecting the arrival of the caregiver in the patient's room in block 386).

Still referring to FIG. 6, the alert management compute device 150 may update the alert suppression criteria as a function of (e.g., based on) the determined result (e.g., from block 374), as indicated in block 390. In doing so, and as indicated in block 392, the alert management compute device 150 may provide the determined result to a neural network (e.g., in the memory 204 or the data storage 212) for use as training data (e.g., feedback). As indicated in bock 394, if the alert was accepted, the alert management compute device 150 may decrease a suppression value (e.g., a numeric value represented in the alert suppression criteria that affects whether an alert is suppressed) to decrease a likelihood of a subsequent alert of the same type (e.g., sharing a threshold amount of similarity in the alert data and context of the caregivers (e.g., locations, level of fatigue, etc.)) being suppressed. Conversely, if the alert was dismissed, the alert management compute device 150 may increase a suppression value to increase a likelihood of a subsequent alert of the same type being suppressed, as indicated in block 396.

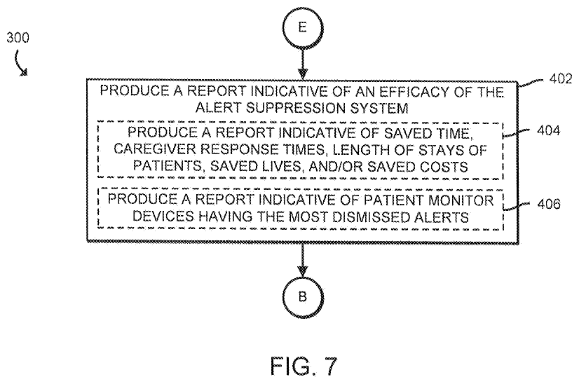

In some embodiments, and as indicated in block 398, the alert management compute device 150 may update the suppression criteria based further on obtained data indicating why the alert was dismissed (e.g., the data from block 378). For example, the obtained data may indicate the specific aspect of the alert data (e.g., that a reported heart rate increase was not indicative of a substantial risk in view of the patient's age) that caused the alert to be dismissed and may be used to more precisely update the alert suppression criteria (e.g., by increasing the suppression value for alerts related to the heart rate for patients of a particular age group, rather than for patients of all ages). As indicated in block 400, the alert management compute device 150 may export the alert suppression criteria (e.g., as a file or other data set) for use in other alert suppression systems (e.g., other installations of the alert management compute device 150 in other clinical environments). Subsequently, the method 300 advances to block 402 of FIG. 7, in which the alert management compute device 150 may produce a report indicative of an efficacy (e.g., effectiveness) of the alert management system 100 (e.g., in sending alerts that warrant caregiver attention and suppressing alerts that would only add to caregiver fatigue).

Referring now to FIG. 7, in producing a report, the alert management compute device 150 may produce a report indicative of saved time, caregiver response times, length of stays of patients in the clinical environment, saved costs (e.g., estimated based on reduced lengths of stay, etc.), as indicated in block 404. For example, the alert management compute device 150 may produce a report in which any of the above factors are represented in trend lines, showing increases or decreases in the factor(s) over time. Additionally or alternatively, the alert management compute device 150 may produce a report indicative of patient monitor devices 110 having the most dismissed alerts, as indicated in block 406. Such a report may be useful in identifying problematic patient monitor devices 110. For example, a particular breathing monitor (e.g., identified by manufacturer and model number) may produce significantly more duplicative or otherwise unwarranted alerts than breathing monitors of other makes or models and, as such, should be suppressed more than other breathing monitors. In the illustrative embodiment, the method 300 subsequently loops back to block 404 of FIG. 3, in which the alert management compute device 150 potentially receives additional alert data (e.g., indicative of another alert). Although the operations of the method 300 are shown in FIGS. 3-7 in a particular order, it should be understood that the operations may be performed in a different order and/or concurrently (e.g., receiving alert data from a patient monitor device 110 while concurrently sending an alert relating to a different patient to one or more caregiver notification devices 120).

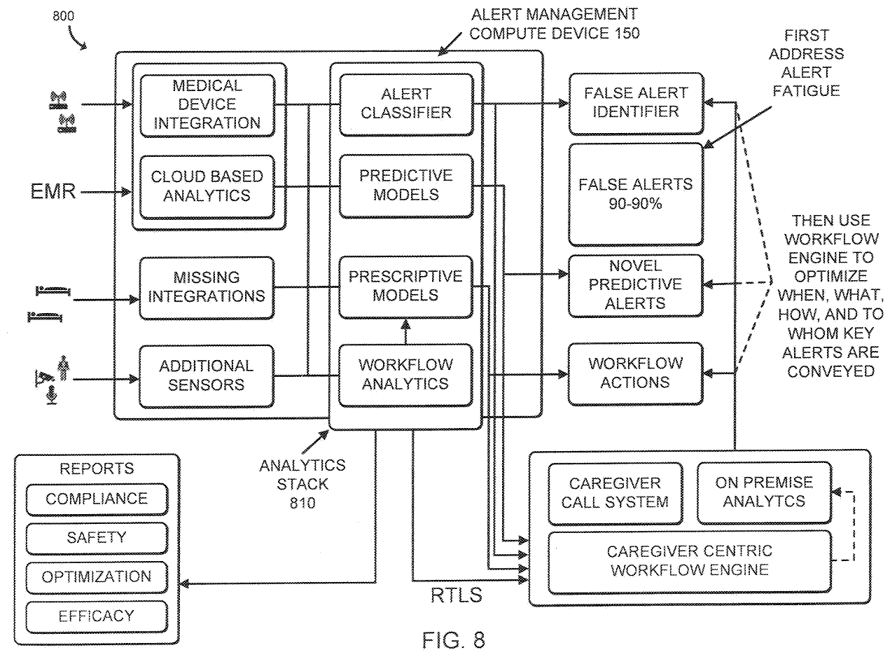

Referring now to FIG. 8, the system 100 may implement a workflow 800. As shown, data from a medical device integration system (e.g., Excel Medical Device Integration (MIDI) from Hillrom of Batesville, IN), cloud based analytics, and/or other medical devices and sensors are received by an analytics stack 810, implemented with the alert management compute device 150. The analytics stack 810 includes an alert classifier (e.g., to identify “false alerts” which may be embodied as alerts that the alert management compute device 150 has determined to suppress, such as according to the method 300). The analytics stack 810 also includes predictive models which may be embodied as functions to identify whether the incoming data (e.g., alert data) is indicative of a patient status that may not be represented by data from a single patient monitor device 110 and/or may be indicative of a patient status that is likely to occur within a defined time frame. Additionally, the analytics stack 810 includes prescriptive models which may be embodied as models used to determine a responsive action, based on one or more rules. Further, the analytics stack 810 includes workflow analytics, which may be embodied as one or more functions to analyze tasks that caregivers are presently assigned to, caregiver schedules, caregiver energy levels, and/or other factors related to the workflow of caregivers in the clinical environment 170. The system 100 first addresses alert fatigue among the caregivers (e.g., by suppressing false alerts). Of the remaining alerts that are not suppressed, the system 100 utilizes a workflow engine (e.g., also implemented by the alert management compute device 150) to determine when, what, how, and to whom alerts are to be conveyed. Corresponding alerts may be conveyed to caregivers via a caregiver call system (e.g., the caregiver notification devices 120 of FIG. 1). Output from the analytics stack 810 also includes report(s) (e.g., a report from block 402 of FIG. 7) with data indicative of compliance, safety, optimization, and/or efficacy of the system 100.

In view of the foregoing, the system 100, depending on the embodiment, may ingest static and streaming output from relevant ward-level medical devices at the raw data level. Additionally or alternatively, the system 100 may ingest information extracted from information technology (IT) systems such as electronic medical records (EMR), personal health records (PHR), and/or other systems containing personal health information. The system 100 may additionally or alternatively ingest information from an admissions, discharge, transfer system (ADT), patient, device, caregiver nurse call and locator services, an internet of things (IOT) sensor network through standard (HL7) or proprietary application programming interfaces (APIs). In some embodiments, the system 100 may automatically detect disruptive third-party interface changes and adapt correspondingly. The system 100 may, in some embodiments, normalize and integrate disparate data sources on premise or in the cloud into the alert management compute device 150. The system 100, in the illustrative embodiment, also executes a multitude of personalized risk algorithms in real-time to become the single alert source for all caregivers in the ward or ward(s) (e.g., the clinical environment 170).

As describe above, the alert management compute device 150 may operate as a central hub that integrates and communicates the following to designated mobile platforms and/or EMR: Intelligent task delegation to specific person(s) by considering caregiver context (e.g., distance to patient, current task cognitive and physical demands, caregiver workload, activity, exertion level, energy level phases (e.g., time of day, time from shift start, etc.), caregiver expertise/certification, distance of caregivers to patients, priority of current or next task relative to priority of a new/high priority task, patient nurse call request history including caregiver assessment of importance of each nurse call, patient location (e.g., in bed, toilet, chair), patient fall/exit alerts, patient activity history and trend, patient toileting history and trends, patient sleep history, patient schedule history and future visits with phlebotomist, physical therapist, respiratory therapist, etc.) etc.). In some embodiments, the caregiver activity/energy level can be derived from body-worn sensors (e.g., a smartwatch). The system 100 may provide a notification of an alert, the alert type, and the patient location, and provide patient finder assistance consistent with alert preferences of the target ward (or caretakers).

The system 100 may analyze trends of the actionable personalized patient risk (e.g., predictive analytics). Additionally or alternatively, and as described above, the system 100 may provide easily accessible and interpretable rationale for the alerts (e.g., interpretability). In the illustrative embodiment, the system 100 informs the caregiver of the next best action to take if prompted by the caregiver (e.g., prescriptive analytics). In some embodiments, alerts can be silent (haptic), checked on a mobile device, and/or interpreted through a digital whiteboard. When on a mobile device, the caregiver may make and receive voice activated calls or use messaging related to alerts (to caregivers, order physician, certified nurse anesthetist, patient). The calls or text messages may be preserved and linked to the alert. The system 100, in some embodiments, notifies other alerted caregivers that the patient is cared for in real-time, and provides data regarding the presence and departure of family member(s). Alert sensitivity can be customized at the level of recipient, recipient's context, locations and devices (real time location services, video integrations, etc.). In some embodiments, the system 100 provides a mechanism (voice or other (real time location service, proximity sensor, camera based intelligent monitoring)) for a caregiver to directly or indirectly indicate that they have arrived at the patient bed.

As discussed above, the system 100 may also execute a continuous learning algorithm that enables caregivers to provide feedback on their clinical context, alert accuracy, sensitivity and reasoning during or after muting/unmuting of alerts (rules-based or machine learning reinforced learning). The system 100 may, in some embodiments, have the capability to clone a learned configuration of champion users from one clinician to another or one customer to another. As such, configurations can be crowd-sourced to a single or multiple site(s). The system 100, in some embodiments and as discussed above, may generate automated reports and keep a tab of total and individual saved time, trending of reaction times, estimate of saved lives, prevented injuries (falls, sepsis, etc.), trends in length of stay per injury type, and provide saved cost estimates. The continuously evolving alert settings may be centrally kept and reused to expedite a subsequent customer deployment and to enable sharing of best of breed customer settings with other users of the system 100 (e.g., installations of the system 100 in other clinical environments). The learned configurations, in some embodiments, can be frozen, and imaged at certain times to enable resetting the configuration back to a time instance.

In some embodiments, the system 100 allows comparison of key metrics from one configuration set to another to help health care facilities make decisions. As discussed above, the system 100 integrates with digital whiteboards, an on premise analytics and notification platform (e.g., Hillrom Voalte®), mobile phones, and other devices. In some embodiments, the system 100 enables integration of risk prediction algorithms from external parties so the notification system can prioritize across all potential caregiver actions. The system 100 may perform task prioritization that considers all past and future tasks for a caregiver, a timeline in a caregiver's shift (e.g., more alerts earlier in shift or after meal break or other break including non-work chats), and mental and physical burden of tasks. As indicated above, a caregiver that receives a task assignment can reject the task (e.g., an alert), and a task engine in the system 100 may reassigns the task to another caregiver. In some embodiments, the system 100 can require that only a specific caregiver complete a task type, thus precluding a caregiver's option to reject the task. In some embodiments, other limits can be set, such as the number of rejections allowed by a caregiver (or the aggregate total of all caregivers) in a shift or other set time period.

The system 100 may be used for remote patient monitoring. That is, the alert management compute device 150 may integrate home-based remote monitoring devices and issue high accuracy alerts for the patient, home caregivers, hospital caregivers, emergency room, and ambulance emergency medical technicians as appropriate. The system 100 may a track patient's health from the home perspective by monitoring devices at home (e.g., a continuous positive airway pressure (CPAP) machine, home scale, pulse oximeter, walker at home), family doctor medical records, portable vital measurement devices (e.g., from a smart watch, etc.). As such, the system 100 may allow relevant information about a patient at home to be available online, or to be transferred to destination a hospital upon admission to the hospital. The system 100 may also provide caregivers a summary of the patient story and current risk levels before entering the hospital. In some embodiments, the system 100 enables a caregiver to provide configuration of alerts at the bed side or remotely through vocal input, a touch based mobile device, or through a standard personal computer interface. In doing so, the system 100 may ask the caregiver or provider to validate the captured alert rules, their duration, and mode. In reducing inactionable patient alerts (false alerts), the system 100 may reduce such alerts at: (1) signal acquisition, that is, the interface between patient and medical devices; (2) alert generation, that is, the algorithms that determine an alert situation; (3) alert validation, that is, determining whether the alert is actually valid; and/or (4) integration of multiple alerts, for example, from different devices, into a smaller number of alerts. The system 100, as discussed above, may also perform alert reduction by performing buffering and reasoning of repeating alerts of the same type for the same patient, relating to the same incident (e.g., consolidating those alerts into a single alert). That is, in operation, the system 100 may operate to ensure that caregivers do not receive more than one notification about the same type of anomaly for the same patient within a minimum interval of time (MINI) that is based on clinical context, personalized to each patient based on their comorbidities, demographics, lab results, and/or other factors. The minimum interval of time can also be further customized by the caregiver, with the caveat that the system 100 may impose limits to prevent jeopardizing patient health.

While certain illustrative embodiments have been described in detail in the drawings and the foregoing description, such an illustration and description is to be considered as exemplary and not restrictive in character, it being understood that only illustrative embodiments have been shown and described and that all changes and modifications that come within the spirit of the disclosure are desired to be protected. There exist a plurality of advantages of the present disclosure arising from the various features of the apparatus, systems, and methods described herein. It will be noted that alternative embodiments of the apparatus, systems, and methods of the present disclosure may not include all of the features described, yet still benefit from at least some of the advantages of such features. Those of ordinary skill in the art may readily devise their own implementations of the apparatus, systems, and methods that incorporate one or more of the features of the present disclosure.

1-10. (canceled)

11. A method for providing an alert in a healthcare system, the method comprising:
monitoring a plurality of patient monitor devices communicatively coupled to a healthcare facility network and operable to monitor one or more statuses associated with a corresponding patient,
sending alert data to the healthcare facility network if a respective status is indicative of an alert condition,
tracking caregiver movement of a plurality of caregivers from a patient position by using a location tracking system,
sensing an activity level of the plurality of caregivers by using a body sensor transported by each the plurality of caregivers,
operating a compute device to use a model of caregiver fatigue for each of the plurality of caregivers to determine a caregiver fatigue level for each of the plurality of caregivers,
selecting a subset of caregivers who can receive the alert based on the caregiver fatigue level,
prioritizing from the subset of caregivers, at least one caregiver for receiving the alert, and
reassigning the alert if the alert is rejected.

12. The method of claim 11, wherein the model of caregiver fatigue includes a work schedule of the at least one caregiver.

13. The method of claim 11, wherein the model of caregiver fatigue includes an energy level of the at least one caregiver.

14. The method of claim 13, wherein the energy level of the at least one caregiver is based on time of day, time from start of shift of the at least one caregiver, or time since last break taken by the at least one caregiver.

15. The method of claim 11, wherein the model of caregiver fatigue includes time periods between previous alerts sent to and responses received from the at least one caregiver.

16. The method of claim 11, wherein the alert is rejected based on the caregiver fatigue level of the at least one caregiver.

17. The method of claim 11, wherein the method further comprises selecting the subset of caregivers to receive the alert as a function of a proximity of the plurality of caregivers to a patient associated with the patient monitor device that produced the alert data.

18. The method of claim 11, wherein the method further comprises selecting the subset of caregivers to receive the alert based on a medical need of a patient associated with the patient monitor device that produced the alert data.

19. The method of claim 11, wherein the method further comprises selecting the subset of caregivers to receive the alert based on an education background or threshold number of years of experience of each of the plurality of caregivers.

20. A method for managing a task, the method comprising:
identifying the task;
prioritizing a caregiver based in part on a dynamic attribute that is associated with the caregiver;
selecting the caregiver as a recipient of the task based in part of the prioritizing;
assigning the task to the recipient; and
reassigning one or more tasks previously assigned to the recipient to another caregiver.

21. The method of claim 20, wherein the caregiver dynamic attribute includes a work schedule of a plurality of caregivers.

22. The method of claim 20, wherein the caregiver dynamic attribute includes an energy level of a plurality of caregivers.

23. The method of claim 20, wherein the caregiver dynamic attribute includes proximity of a plurality of caregivers to the patient.

24. The method of claim 20, wherein the caregiver dynamic attribute includes a medical need of the patient that produced the alert, education background of a plurality of caregivers, or threshold number of years of experience of a plurality of caregivers.

25. An alert management system for use in a healthcare facility, the alert management system comprising
a healthcare facility network;
a plurality of patient monitor devices communicatively coupled to the network and operable to send alert data associated with a patient to the network,
a location tracking system coupled to the network, the location tracking system configured to track caregiver movement of at least one caregiver from a patient position;
a body sensor transported by the at least one caregiver and configured to sense an activity level of the at least one caregiver; and
a compute device coupled to the network, the compute device configured to
receive the alert data, and, in response, generate an alert to be provided to a caregiver notification device of the at least one caregiver out of a plurality of caregivers unless an alert suppression criteria is satisfied, wherein the caregiver notification device is configured to permit the at least one caregiver to reject the alert provided, and wherein the alert suppression criteria is a function of activity level data from the body sensor;
suppress, in response to a determination that the alert suppression criteria is satisfied, the alert from being provided to the caregiver notification device of the at least one caregiver; and
generate an automated report that summarizes saved time and improved caregiver response times.

26. The alert management system of claim 25, wherein the compute device is further configured to determine a result of providing the alert to the caregiver and to provide the result to a neural network in the healthcare facility network as training data such that the alert suppression criteria is updated for use in future determinations of alert-related fatigue.

27. The alert management system of claim 25, wherein the compute device is configured to determine a caregiver fatigue level based on a model of caregiver fatigue for the at least one caregiver based on the activity level sensed by the body sensor, and wherein the caregiver fatigue level is used for the determination of whether the alert suppression criteria is satisfied.

28. The alert management system of claim 25, wherein the compute device is configured to use a model of caregiver fatigue for each of the plurality of caregivers to determine a caregiver fatigue level and select a subset of the plurality of caregivers to receive the alert, and wherein the compute device is configured to exclude from the subset, any caregivers with the caregiver fatigue level above a reference amount.

29. The alert management system of claim 25, further comprising a visual sensor for determining caregiver fatigue level, wherein the caregiver fatigue level is used for the determination of whether the alert suppression criteria is satisfied.

30. The alert management system of claim 25, wherein the alert is routed to the at least one caregiver based on caregiver distance of the at least one caregiver from the patient position, and wherein the alert is sent to the caregiver notification device of the at least one caregiver if the caregiver distance from the patient position is within a predefined range.
